(19) United States
(12) Patent Application Publication
(10) Pub. No.: US 2013/0251804 A1
(43) Pub. Date: Sep. 26, 2013

(54) METHOD TO IMPROVE THE SAFETY OF HANDLING OF HIGH POTENCY DRUGS IN SOLID DOSAGE FORMS WITHOUT CHANGING THEIR EFFICACY

(71) Applicant: EMPHASCIENCE, INC., Pittsford, NY (US)

(72) Inventors: Rebanta Bandyopadhyay, Webster, NY (US); Susen Werle, Webster, NY (US)

(73) Assignee: Emphascience, Inc., Pittsford, NY (US)

(21) Appl. No.: 13/848,493

(22) Filed: Mar. 21, 2013

Related U.S. Application Data
(60) Provisional application No. 61/613,639, filed on Mar. 21, 2012.

(51) Int. Cl. A61K 9/28; A61K 31/58

(52) U.S. Cl. CPC A61K 9/2866; A61K 31/58
USPC 424/480; 514/176

(57) ABSTRACT

A method to improve the safety of handling of drug substances that are dispensed as solid oral dosage forms is described that does not alter the drug-release profile and the therapeutic efficacy of the pharmaceutical product.

### CROSS-REFERENCE TO RELATED APPLICATIONS

This application claims priority to and the benefit of U.S. provisional patent application Ser. No. 61/613,639 (filed Mar. 21, 2012), which application is incorporated herein by reference in its entirety.

### FIELD OF THE INVENTION

The subject matter disclosed herein relates to pharmaceutical products. More particularly, this disclosure is in the area of improving safety of handling and use of solid oral dosage forms, such as, tablets and capsules.

### BACKGROUND

Due to the relatively lower costs associated with manufacturing and packaging and for improved chemical stability, pharmaceutical substances are most often dispensed as solid oral dosage forms, such as tablets and packaged in bulk in glass or high density polyethylene bottles. While the tablets are typically designed to have the strength and breakage characteristics suited to their handling and use, often the bulk packaging of tablets leads to attrition and formation of dust that may contain trace amounts of the drug substance along with the dust of other excipients added to the tablet. It is also possible that nurses, adult family members of a patient and lay caregivers who may not be equipped and trained to handle pharmaceutical active ingredients, may open the packages to fetch the tablet dose with bare hands or fingers and offer the tablet to patients that suffer from a debilitating disease and, thus, inadvertently expose themselves to the dusts containing pharmacologically active drug substances. For high potency medicinal substances or drugs that carry a high burden of risk and are intended only for those afflicted with a serious disease or for those active ingredients that have gender or age-related toxicities, the handling of a tablet dosage form might be the cause for inadvertent and undesirable toxicity.

### SUMMARY OF THE INVENTION

The method comprises of application of a protective or barrier layer that is significantly devoid of the active ingredient over the oral dosage form, such as a tablet, to prevent inadvertent exposure to the drug during the course of handling it with bare hands. In addition to preventing the surface erosion or surface wear of particles of the active ingredient on to the hands of anyone handling it, such protective or barrier layer combined with a rounded shape of the tablet or other oral dosage form prevents the generation of dust within the package container containing such tablets in bulk, as well. While the barrier layer prevents migration of the drug and exposure to the handler, the release profile of the drug from the tablet into the physiological media and, subsequently, its absorption following oral administration remains unaffected. More particularly, the composition of the protective layer and the medium of application of the protective layer, as well as the method of such application, prevents migration of the active ingredient to the surface of the tablet during the process of manufacturing and during storage.

As a particular example, the drug product ZYTIGA® containing the active ingredient, abiraterone acetate, is indicated for the treatment of metastatic castration-resistant prostate cancer (CRPC) in combination with prednisone. Each high-density polyethylene bottle of the product contains 120 white to off-white, oval 250 mg tablets. ZYTIGA® is not indicated in women and children. Based on its mechanism of action, abiraterone acetate, may harm a developing fetus. Therefore, the package insert for ZYTIGA® specifies that women who are pregnant or women who may be pregnant should not handle ZYTIGA® without protection, e.g., gloves. Although it is not known, if abiraterone acetate is excreted in human milk, because of the high lipophilicity of the drug substance and potential for serious adverse reactions in nursing infants in case it is excreted in human milk, the consequences of inadvertent exposure to the drug substance can be serious for both, mother and child. In fact, the phase III clinical trials of the product almost exclusively involved male patients that were 65 years and older. Considering the recommended dose of 1000 mg daily, which involves the administration of up to 4 of the 250 mg tablets, and the typical age of a patient and the target disease condition, the involvement of a family member in the administration of the drug is quite possible. Hence a strategy to improve the safety of handling of ZYTIGA® that does not significantly alter the drug-release profile and the therapeutic efficacy will be very useful in preventing inadvertent exposure to women and children.

### BRIEF DESCRIPTION OF THE DRAWINGS

The present invention is disclosed with reference to the accompanying drawing(s), wherein:

FIG. 1 shows the position a barrier film around an ovoid-shaped table.

The examples set out herein illustrate several embodiments of the invention but should not be construed as limiting the scope of the invention in any manner.

### DETAILED DESCRIPTION

Tablet dosage forms are designed to withstand common handling and shipping conditions without breakage or much attrition and, yet, disintegrate and dissolve at the site of absorption to deliver the drug substance for systemic uptake following administration to a patient. Excipients that increase the compressibility and adhesion of the active substance are added to the powder mixture before compression to impart strength, while so-called disintegrants added to the tablet formulation assist in the breakage and release of the drug into the systemic fluids following administration. For brittle and semi-brittle tablets that are not individually protected, limited attrition can occur at the edges due to localized stresses leading to the formation of radial and lateral cracks under the surface. The sub-surface lateral cracks are considered to be the primary cause for material removal by attrition. The risk of inadvertent exposure to a drug substance to an individual other than the patient is high when, due to such attrition, the drug substance could “chip off” either during direct handling or due to attrition during shipping and storage.

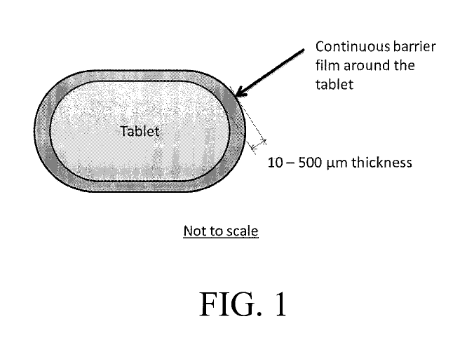

A method to avoid such exposure without changing the formulation or the rate of drug release for absorption is to form a thin but effective barrier layer of a protective film around the tablet as shown in FIG. 1 that is completely free of the drug substance, resists chipping and erosion, and yet, completely and immediately dissolves in contact with physiological media so as not to influence the drug release rate upon administration.

In addition to the film, a rounded ovoid shape of the tablet will reduce the number and length of edges that are more prone to surface erosion of the active ingredient during handling, shipping and storage.

One such method of forming the film is to apply a thin but robust coat of a polymer solution or suspension, or a combination of polymers in solution or suspension in a suitable medium and in a suitable ratio over the surface of the tablet by various conventional spray coating processes, whereby a solution of the polymer(s) is sprayed on the tablets and then dried to create an uninterrupted barrier layer of about 10 to 500 micrometers thickness on the surface of the tablet. In one embodiment, the thickness of the polymer layer is between 20 micrometers and 150 micrometers. The polymer solution or suspension may also contain other commonly added components, such as, plasticizers to impart optimum mechanical properties to the polymer film-coat.

Such a coating process will involve the simultaneous application of heat and increased airflow to facilitate the quick drying of the tablet surface, such that the liquid medium does not penetrate the tablet to any extent to alter the composition and characteristics of the core tablet or to facilitate the migration of the active ingredient through the film. In one embodiment, the liquid medium is removed within one minute of application to prevent penetration of the activity ingredient into the coating. In another embodiment, the liquid medium is removed within thirty seconds of application to prevent penetration of the activity ingredient into the coating.

The choice of film-forming polymers for coating the tablets may include various cellulosic derivatives, such as, hypromellose (hydroxypropylmethyl cellulose), hydroxyethyl cellulose, hydroxylethylmethyl cellulose, carboxymethyl cellulose and its salts and esters, ethyl cellulose, hydroxypropyl cellulose, polyethylene glycol and other glycol derivates, polyethylene oxide, various sugars, such as, xylitol, confectioner's sugar, sucrose, pregelatinized starch, pullulan, polydextrose, maltodextrin, maltitol, isomalt, glucose, as well as, other miscellaneous coating agents, such as, chitosan, cellulose, corn syrup solids, ethylene glycol and vinyl alcohol grafted copolymer, gelatin, colophony, polyvinyl acetate, polyvinyl acetate phthalate, polyvinyl alcohol, polyvinyl pyrrolidone (povidone)

The choice of typical plasticizers can include polyols, such as, glycerol, propylene glycol, PEG (polyethylene glycol), organic esters, such as phthalate esters, dibutyl sebacete, citrate esters, triacetin, and oils/glycerides, such as, castor oil, acetylated monoglycerides, and fractionated coconut oil.

In the particular case cited for abiraterone acetate (ZYTIGA®), the use of water soluble polymers with more polar characteristics and water as a film-coating medium would be used to ensure that the lipophilic and non-polar drug substance is prevented from dissolving or migrating into and through the layer to the surface of the tablet during manufacturing and storage.

Other suitable examples of active ingredients include hormone-altering drugs (e.g. finasteride, dutasteride) and drugs that are believed to cause birth defects (e.g. misoprostol) and other anti-Parkinson's disease, antineoplastic, immunosuppressant drugs that have the possibility of causing severe side-effects, such as, azathioprine, everolimus, mercaptopurine, methotrexate, mifepristone, mitotane, rasgiline, thioguamine, and toremefine citrate. In one embodiment, the active ingredient is pharmaceutically active. In one such embodiment, the pharmaceutical activity is pharmaceutical activity in a human being. In another embodiment, the pharmaceutical activity is pharmaceutical activity in a domesticated animal.

For the same reasons cited above, the choice of plasticizer will be restricted to more polar components to prevent the dissolution and migration of the active ingredient, abiraterone acetate, into the barrier film.

The foregoing written description of the invention enables one of ordinary skill to make and use what is considered presently to be the best mode thereof. Those of ordinary skill in the trade will also understand and appreciate the existence of variations, combinations, and equivalents of the specific embodiments, methods, and examples herein and, hence, the invention should not be limited by the specific embodiment of the method and examples, but by all embodiments and methods within the scope and spirit of the invention as stated.

What is claimed is:

1. A dosage form comprising:
a pharmaceutically active ingredient in a the form of a rounded pill;
a coating encapsulating the pharmaceutically active ingredient, the coating being between 10 and 500 micrometers thickness made from a water-soluble polymer selected from the group consisting of hydroxypropylmethyl cellulose, hydroxyethyl cellulose, hydroxylethylmethyl cellulose, carboxymethyl cellulose and its salts, ethyl cellulose, hydroxypropyl cellulose, and polyethylene glycol.

2. The dosage form of claim 1 wherein the pharmaceutically active ingredient is abiraterone acetate.

3. The dosage form of claim 1 wherein the coating is between 20 and 150 micrometers thick.

4. The dosage form of claim 1, the coating further comprising a plasticizer selected from the group consisting of a polyol, an organic ester and a glyceride.

5. The dosage form of claim 4, wherein the plasticizer is a polyol selected from the group consisting of glycerol, propylene glycol and polyethylene glycol (PEG).

6. The dosage form of claim 4, wherein the plasticizer is an organic ester selected from the group consisting of phthalate ester, dibutyl sebacete, citrate ester and triacetin.

7. The dosage form of claim 4, wherein the plasticizer is a glyceride selected from the group consisting of castor oil, acetylated monoglyceride, and fractionated coconut oil.

8. A dosage form comprising:
a pharmaceutically active ingredient in a the form of a rounded pill;
a coating encapsulating the pharmaceutically active ingredient, the coating being between 10 and 500 micrometers thickness made from a water-soluble cellulosic polymer selected from the group consisting of hydroxypropylmethyl cellulose, hydroxyethyl cellulose, hydroxylethylmethyl cellulose, carboxymethyl cellulose and its salts, ethyl cellulose and hydroxypropyl cellulose.

9. The dosage form of claim 1 wherein the pharmaceutically active ingredient is abiraterone acetate.

10. The dosage form of claim 1 wherein the coating is between 20 and 150 micrometers thick.

11. A method for forming a dosage form, the method comprising:
coating a rounded pill with a water-soluble polymer in a liquid medium to form a coating, the rounded pill comprising a pharmaceutically active ingredient;
removing the liquid medium by drying before the active ingredient penetrates into the water-soluble polymer;
the coating encapsulating the active ingredient, the coating being between 10 and 500 micrometers thickness, and the water-soluble polymer being selected from the group consisting of hydroxypropylmethyl cellulose, hydroxyethyl cellulose, hydroxylethylmethyl cellulose, carboxymethyl cellulose and its salts, ethyl cellulose, hydroxypropyl cellulose, and polyethylene glycol.

12. The method as recited in claim 11, wherein the step of removing the liquid is completed within one minute of the step of coating the rounded pill.

13. The method as recited in claim 11, wherein the water-soluble polymer is a water-soluble cellulosic polymer selected from the group consisting of hydroxypropylmethyl cellulose, hydroxyethyl cellulose, hydroxylethylmethyl cellulose, carboxymethyl cellulose and its salts, ethyl cellulose and hydroxypropyl cellulose.
